(19) United States
(12) Patent Application Publication
(10) Pub. No.: US 2010/0125319 A1
(43) Pub. Date: May 20, 2010

(54) CELL-REPELLING POLYMERIC ELECTRODE HAVING A STRUCTURED SURFACE

(76) Inventor: Torsten Scheuermann, Munich (DE)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/576,918

(22) Filed: Oct. 9, 2009

Related U.S. Application Data
(60) Provisional application No. 61/116,584, filed on Nov. 20, 2008.

(51) Int. Cl. A61N 1/05; H01R 43/00

(52) U.S. Cl. 607/116; 29/825

(57) ABSTRACT

The embodiments herein relate to a coated electrode including a structured surface and a conductive layer and a method of making the same. The various electrode embodiments can include a surface topography that minimizes tissue attachment and thus facilitates removal of the electrode.

### CROSS-REFERENCE TO RELATED APPLICATION

This application claims priority under 35 U.S.C. §119 to U.S. Provisional Application No. 61/116,584, filed on Nov. 20, 2008, entitled “Cell-Repelling Polymeric Electrode Having a Structured Surface,” which is incorporated herein by reference in its entirety for all purposes.

### TECHNICAL FIELD

This invention relates to body implantable medical devices, and more particularly, to implantable electrodes for sensing electrical impulses in body tissue or for delivering electrical stimulation pulses to an organ, for example, for pacing the heart.

### BACKGROUND

Various types of medical electrical leads for use in cardiac rhythm management systems are known. Such leads are typically extended intravascularly to an implantation location within or on a patient's heart, and thereafter coupled to a pulse generator or other implantable device for sensing cardiac electrical activity, delivering therapeutic stimuli, and the like. The leads are desirably highly flexible to accommodate natural patient movement, yet also constructed to have minimized profiles. At the same time, the leads are exposed to various external forces imposed, for example, by the human muscular and skeletal system, the pulse generator, other leads, and surgical instruments used during implantation and explantation procedures. There is a continuing need for improved lead designs.

### SUMMARY

The present invention, in one embodiment, is a medical electrical lead comprising a flexible lead body defining at least one longitudinal lumen therethrough, a conducting wire extending through the at least one lumen, a connector coupled to the lead body for mechanically and electrically coupling the lead to an implantable pulse generator device, and an electrode. The electrode includes an electrode body including a structured surface, and a conductive coating disposed on the structured surface. The conductive coating is electrically coupled to the at least one conducting wire. In one alternative, the structured surface has pillars. In another alternative, the conductive coating has an adhesion layer disposed on the structured surface and an external coating disposed over the adhesion layer.

In another embodiment, the present invention is a method of making an electrode for a medical electrical lead of the type having a flexible lead body and at least one electrical conducting wire therein. The method comprises forming an electrode body, forming a structured surface on the electrode body, applying an adhesion layer to the structured surface, and applying a conductive external coating to the adhesion layer.

In yet another embodiment, the present invention is an electrode for use on an implantable medical electrical lead having at least one conductive member. The electrode comprises an electrode body comprising a structured surface and a conductive coating disposed on the structured surface. The conductive coating has a surface topography configured to exhibit hydrophobic behavior, and is configured to be electrically coupled to the lead conductive wire.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic drawing of a cardiac rhythm management system including a pulse generator coupled to a pair of medical electrical leads deployed in a patient's heart, according to one embodiment.

FIG. 2 is a perspective view of one of the leads shown in FIG. 1, according to one embodiment.

FIG. 3A is a schematic drawing of an electrode having a body and a structured surface and conductive coating, according to one embodiment.

FIG. 3B is another, expanded view of the schematic drawing of the electrode of FIG. 3A, according to one embodiment.

FIG. 4 is an image of exemplary pillar-like structures of a structured surface, according to one embodiment.

FIG. 5 is an image of exemplary rice grain structures of an external coating, according to one embodiment.

FIG. 6 is a cross-sectional view of an electrode, according to one embodiment.

### DETAILED DESCRIPTION

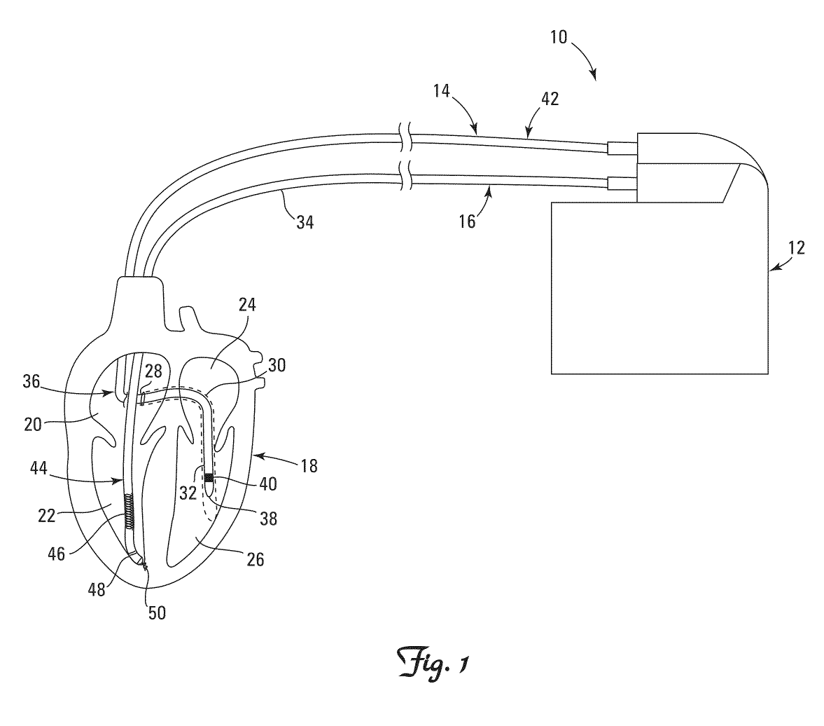

The various embodiments disclosed herein relate to a medical electrical lead having an electrode having a structured surface and a conductive coating and related methods of making the lead. The leads according to the various embodiments of the present invention are suitable for sensing intrinsic electrical activity and/or applying therapeutic electrical stimuli to a patient. Exemplary applications include, without limitation, cardiac rhythm management (CRM) systems and neurostimulation systems. For example, in exemplary CRM systems utilizing pacemakers, implantable cardiac defibrillators, and/or cardiac resynchronization therapy (CRT) devices, the medical electrical leads according to embodiments of the invention can be endocardial leads configured to be partially implanted within one or more chambers of the heart so as to sense electrical activity of the heart and apply a therapeutic electrical stimulus to the cardiac tissue within the heart. Additionally, the leads formed according to embodiments of the present invention may be particularly suitable for placement in a coronary vein adjacent to the left side of the heart so as to facilitate bi-ventricular pacing in a CRT or CRT-D system. Still additionally, leads formed according to embodiments of the present invention may be configured to be secured to an exterior surface of the heart (i.e., as epicardial leads). FIG. 1 is a schematic drawing of a cardiac rhythm management system 10 including a pulse generator 12 coupled to a pair of medical electrical leads 14, 16 deployed in a patient's heart 18, which includes a right atrium 20 and a right ventricle 22, a left atrium 24 and a left ventricle 26, a coronary sinus ostium 28 in the right atrium 20, a coronary sinus 30, and various coronary veins including an exemplary branch vessel 32 off of the coronary sinus 30.

According to one embodiment, as shown in FIG. 1, lead 14 includes a proximal portion 42 and a distal portion 36, which as shown is guided through the right atrium 20, the coronary sinus ostium 28 and the coronary sinus 30, and into the branch vessel 32 of the coronary sinus 30. The distal portion 36 further includes a distal end 38 and an electrode 40 both positioned within the branch vessel 32. The illustrated position of the lead 14 may be used for delivering a pacing and/or defibrillation stimulus to the left side of the heart 18. Additionally, it will be appreciated that the lead 14 may also be partially deployed in other regions of the coronary venous system, such as in the great cardiac vein or other branch vessels for providing therapy to the left side or right side of the heart 18.

In the illustrated embodiment, the electrode 40 is a relatively small, low voltage electrode configured for sensing intrinsic cardiac electrical rhythms and/or delivering relatively low voltage pacing stimuli to the left ventricle 26 from within the branch coronary vein 32. In various embodiments, the lead 14 can include additional pace/sense electrodes for multi-polar pacing and/or for providing selective pacing site locations.

As further shown, in the illustrated embodiment, the lead 16 includes a proximal portion 34 and a distal portion 44 implanted in the right ventricle 22. In other embodiments, the CRM system 10 may include still additional leads, e.g., a lead implanted in the right atrium 20. The distal portion 44 further includes a flexible, high voltage electrode 46, a relatively low-voltage ring electrode 48, and a low voltage tip electrode 50 all implanted in the right ventricle 22 in the illustrated embodiment. As will be appreciated, the high voltage electrode 46 has a relatively large surface area compared to the ring electrode 48 and the tip electrode 50, and is thus configured for delivering relatively high voltage electrical stimulus to the cardiac tissue for defibrillation/cardioversion therapy, while the ring and tip electrodes 48, 50 are configured as relatively low voltage pace/sense electrodes. The electrodes 48, 50 provide the lead 16 with bi-polar pace/sense capabilities.

In various embodiments, the lead 16 includes additional defibrillation/cardioversion and/or additional pace/sense electrodes positioned along the lead 16 so as to provide multi-polar defibrillation/cardioversion capabilities. In one exemplary embodiment, the lead 16 includes a proximal high voltage electrode in addition to the electrode 46 positioned along the lead 16 such that it is located in the right atrium 20 (and/or superior vena cava) when implanted. As will be appreciated, additional electrode configurations can be utilized with the lead 16. In short, any electrode configuration can be employed in the lead 16 without departing from the intended scope of the present invention.

The pulse generator 12 is typically implanted subcutaneously within an implantation location or pocket in the patient's chest or abdomen. The pulse generator 12 may be any implantable medical device known in the art or later developed, for delivering an electrical therapeutic stimulus to the patient. In various embodiments, the pulse generator 12 is a pacemaker, an implantable cardiac defibrillator, a cardiac resynchronization (CRT) device configured for bi-ventricular pacing, and/or includes combinations of pacing, CRT, and defibrillation capabilities.

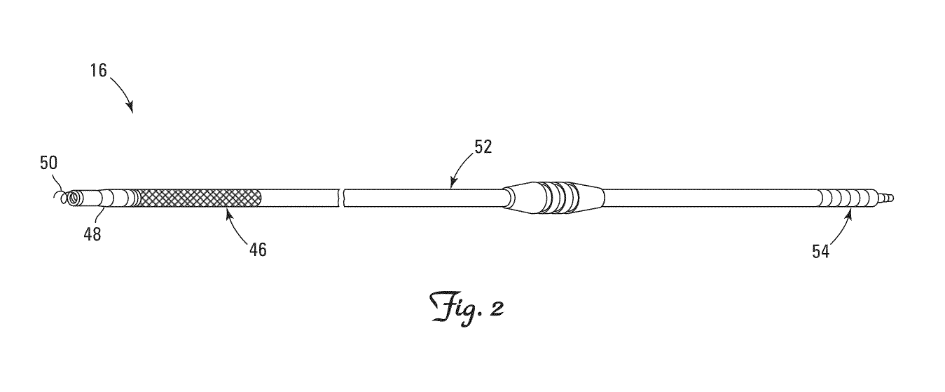

FIG. 2 is a perspective view of the lead 16 shown in FIG. 1. As discussed above, the lead 16 is adapted to deliver electrical pulses to stimulate a heart and/or for receiving electrical pulses to monitor the heart. The lead 16 includes an elongated lead body 52, which may be formed from any polymeric material such as polyamide, polycarbonate, silicone rubber, or the like. Alternatively, the lead body 52 is not polymeric and instead is formed from a metal material such as copper, silver, aluminum, stainless steel (such as, for example, Grade 316L stainless steel), nitinol, CoCr, FePt, or any other metal material that can be used for a lead body.

According to certain implementations in which the lead body is formed from a polymeric material, the polymeric material is stable to a temperature of at least about 100° Celsius. That is, the polymeric material is configured to maintain its integrity up to at least about 100° C. In one aspect, this heat stability allows the polymeric material to withstand the texturing or structuring and coating processes described below. In alternative embodiments in which the lead body is non-polymeric, the metal materials discussed above can withstand much higher temperatures than polymeric materials and thus are stable to temperatures of at least 200° Celsius.

As further shown, the lead 16 further includes a connector 54 operatively associated with the proximal end of the lead body 52. The connector 54 is configured to mechanically and electrically couple the lead 16 to the pulse generator 12, and may be of any standard type, size or configuration. As will be appreciated, the connector 54 is electrically and mechanically connected to the electrodes 46, 48, 50 by way of one or more conducting wires (not shown) within the lead body 52. The conducting wires utilized may take on any configuration providing the necessary functionality. For example, as will be appreciated, the conducting wires coupling the electrodes 48 and/or 50 to the connector 54 (and thus, to the pulse generator 12) may be coiled conductors defining an internal lumen for receiving a stylet or guidewire for lead delivery. Conversely, in various embodiments, the conducting wire to the high voltage electrode 46 may be a multi-strand cable conductor.

According to the various embodiments of the present invention, one or more of the electrodes 46, 48, 50, e.g., the high voltage electrode 46, includes a structured surface configured to exhibit hydrophobic qualities. The hydrophobic qualities advantageously inhibit tissue in growth and/or attachment to the electrode surface. Thus, the electrode configurations according to various embodiments of the present invention provide alternatives to existing techniques for inhibiting tissue adhesion and in growth to electrode surfaces, e.g., ePTFE coatings or wraps.

As will be appreciated, the electrode configurations described herein according to the various embodiments of the present invention may also be utilized for the electrodes of the lead 14 (see FIG. 1) configured for implantation in the coronary venous system, as well as electrodes for other leads such as right atrial and epicardial leads.

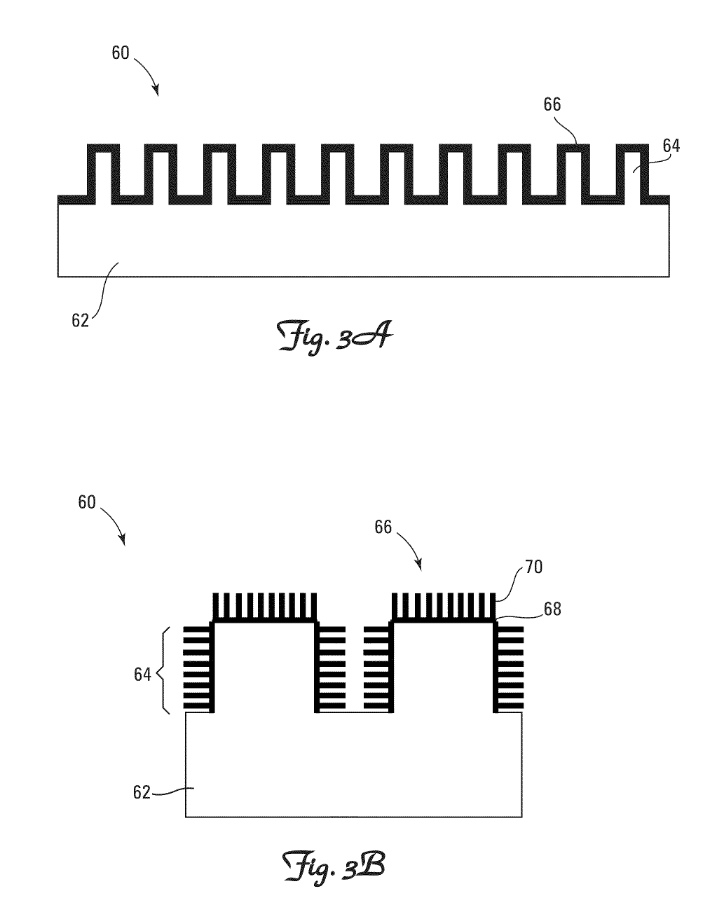

As shown in FIGS. 3A and 3B according to one implementation, an electrode 60 (similar to the electrode in FIGS. 1 and 2) has a polymeric body 62 with a structured surface 64, and a conductive coating 66. In accordance with one embodiment, the conductive coating 66 is made up of an adhesion layer 68 and an external coating 70. Alternatively, as discussed above, the electrode 60 can have a non-polymeric conductive body 62 such as steel or any other conductive material with the structured surface 64 and conductive coating 66.

The “structured surface” 64 is intended to describe any configuration of the body surface resulting from a structuring or texturing process as described herein. Generally, the structured surface 64 can be made up of any known structures or morphology formed on the body that can result in an uneven or non-flat surface topography.

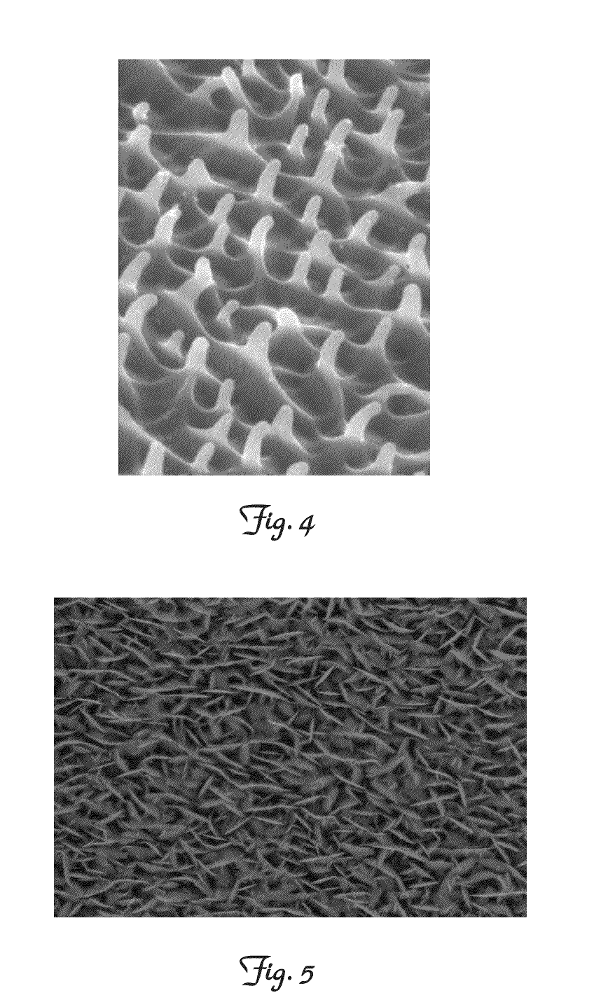

In one implementation, the structured surface 64 is made up of pillar-like structures. One exemplary depiction of such structures is shown in FIG. 4, which is a depiction of an actual structured surface of a polymeric body created by laser etching. Alternatively, the structure surface 64 can be made up of other structures such as wrinkle-like structures or other types of mechanical features such as ridges, holes, voids, steps, etc.

According to one embodiment, the structured surface 64 is created by a laser etching process. In one implementation, the process can be any laser etching process that uses UV wavelengths or picosecond pulses to ablate material. For example, a known dual beam interference procedure could be used. Alternatively, any other known laser-based processes such as laser scanning or pulse techniques can be used. Alternatively, the structure surface 64 can be created using any known printing process such as a hot stamp printing process. In a further alternative, the structured surface 64 is created using a known lithographic process.

The dimensions of the structures of the structured surface 64, in accordance with one implementation, can range from about 10 μm to about 100 μm. That is, the distances between the deepest points and the highest external points along the structured surface 64 vary within that range. Alternatively, the dimensions of the surface 64 can range from about 5 μm to about 50 μm. In a further alternative, the dimensions can range from about 20 μm to about 30 μm.

As mentioned above, the conductive coating 66, according to one embodiment, coats, covers, or is otherwise disposed on the structured surface 64. As also mentioned above, the conductive coating 66 can be made up of two layers: an adhesion layer 68 and an external conductive coating 70.

In one implementation, the adhesion layer 68 is disposed between the structured surface 64 and the external conductive coating 70. According to one embodiment, the adhesion layer 68 can be made up of titanium. Alternatively, the adhesion layer 68 can be comprised of platinum, gold, silver, copper, tantalum, or niobium. In a further alternative, the adhesion layer 68 is made up of any similar known material that can serve as an adhesion layer between the structured surface 64 and the external coating 70.

The adhesion layer 68 can be applied to the structured surface 64 using any known sputtering or deposition process, according to one embodiment. One such process is a chemical vapor deposition (“CVD”) process. The CVD process, in accordance with one implementation, can coat the entire structured surface 64, including small crevices and gaps, with the adhesion layer 68. In fact, according to one embodiment, the CVD process also can coat any connection lumen in communication with the conducting wire lumen (as described below) with the adhesion layer 68.

Exemplary CVD and PVD processes are described in U.S. Pat. No. 6,057,031, which is hereby incorporated herein by reference in its entirety.

In one aspect, the adhesion layer 68 ranges from about 10 nm to about 1000 nm in thickness. Alternatively, the layer 68 ranges from about 10 nm to about 100 nm. In a further alternative, the layer 68 ranges from about 10 nm to about 30 nm.

According to one implementation, the external coating 70 is disposed on or over the adhesion layer 68. According to one embodiment, the adhesion layer 68 adheres the external coating 70 to the structured surface 64.

The external coating 70 can be made up of a ceramic such as iridium oxide, according to one embodiment. The ceramic can be applied to the adhesion layer 68 according to a known application method. According to one embodiment, iridium oxide is applied by a known physical vapor deposition process. Alternatively, the iridium oxide can be applied by a known pulsed laser deposition process. In a further embodiment, the iridium oxide can be applied by any other known method of applying iridium oxide. The IROX™ structure and application methods according to one embodiment are described in some detail in U.S. Pat. Nos. 4,679,572 & 4,762,136.

Various processes can be used to achieve the various textured topographies discussed above. In one embodiment, the coating can be formed via an in-line flat target PVD system (physical vapor deposition using a rectangular chamber with a flat target). One exemplary arrangement of such a system includes a cathode within which resides a target material, such as a ceramic (e.g. IROX) or a ceramic precursor metal (e.g. Ir). The component to be coated is disposed within the chamber. The cylinder also includes a gas, such as argon and oxygen. During the process, plasma formed in the chamber accelerates charged species toward the target, and target material is thereby sputtered from the target and deposited onto the stent. The in-line flat target PVD system is suitable for application of the high frequency bias on the stent to realize the desired coating formation. Another exemplary PVD arrangement is inversed cylindrical PVD system, which is described in U.S. patent application Ser. No. 11/752,772, which is hereby incorporated herein by reference in its entirety.

Another process that could be used herein is high frequency bias sputtering, which is described in Kim et al., Advances in Electronic Materials and Packaging, 2001, EMAP 2001, page 202-207, and further in U.S. Pat. No. 4,897,172, both of which are hereby incorporated herein by reference in their entireties. Further, AC and DC sputtering are further exemplary processes that could be used herein. They are described in 43^{rd} Annual Technical Conference Proceedings, page 81 (2000) and a brochure entitled “Recent developments in inverted cylindrical magnetron sputtering,” available from Isoflux Inc. (Rochester, N.Y.), both of which are hereby incorporated herein by reference in their entireties. Another exemplary process, inverted cylindrical physical vapor deposition, is described further in Siegfried et al., Society of Vacuum Coaters, 39^{th} Annual Technical Conference Proceedings (1996), p. 97; Glocker et al., Society of Vacuum Coaters, 43^{rd} Annual Technical Conference Proceedings-Denver, April 15-20, 2000, p. 81; and SVC: Society of Vacuum Coatings: C-103, An Introduction to Physical Vapor Deposition (PVD) Processes and C-248—Sputter Deposition in Manufacturing, available from SVC 71 Pinion Hill, NE, Albequeque, N.M. 87122-6726, all of which are hereby incorporated herein by reference in their entireties. A suitable cathode system is the Model 514, available from Isoflux, Inc., Rochester, N.Y. Another sputtering technique that could be used herein includes closed loop cathode magnetron sputtering. Pulsed laser deposition could also be employed, and is described in U.S. application Ser. No. 11/752,735, filed May 23, 2007, which is hereby incorporated herein by reference in its entirety. Formation of IROX is further described in Cho et al., Jpn. J. Appl. Phys. 36(I)3B: 1722-1727 (1997), and Wessling et al., J. Micromech. Microeng. 16:5142-5148 (2006), both of which are hereby incorporated herein by reference in their entireties.

The resulting external coating 70 has a certain textured morphology or topography, according to one embodiment. The surface is characterized by its visual appearance, its roughness, and/or the size and arrangement of the particular morphological features such as local maxima.

In one implementation, the textured topography of the external coating 70 is characterized by defined grains and high roughness. For example, in one embodiment, the surface is characterized by definable sub-micron sized grains. FIG. 5 provides one exemplary depiction of such a textured topography. The defined grain, high roughness topography provides a high surface area characterized by crevices between and around spaced grains. In one embodiment, this particular topography is referred to as a “rice grain structure” because the grains resemble rice grains. According to various implementations, the grains have a length ranging from about 50 nm to about 500 nm. Alternatively, the grains have a length ranging from about 100 nm to about 300 nm. In a further embodiment, the grains have a width ranging from about 5 nm to about 50 nm. Alternatively, they have a width ranging from about 10 nm to about 15 nm. The grains have an aspect ratio (length to width) of about 5:1 or more. Alternatively, they have an aspect ratio of from about 10:1 to about 20:1. In one embodiment, the grains overlap in one or more layers. The separation between the grains can range from about 1 nm to about 50 nm. In one embodiment, these types of grains can resemble rice grains and thus the morphology can be referred to as a “rice grain structure.”

In an alternative embodiment, the textured topography of the external coating is characterized by a more continuous surface having a series of globular features separated by striations. In one embodiment, the striations have a width of about 10 nm or less. Alternatively, the striations have a width of about 1 nm or less. In a further alternative, the striations have a width ranging from about 0.1 nm to about 1 nm. According to one implementation, the striations can be generally randomly oriented and intersecting. In one embodiment, the depth of the striations is about 10% or less of the thickness of the coating. Alternatively, the depth of the striations ranges from about 0.1 to about 5% of the thickness of the coating. In accordance with certain embodiments, the globular features separated by striations can resemble the surface of an orange peel.

Alternatively, the textured topography has characteristics ranging between high aspect ratio, definable grains and a more continuous globular surface. For example, in one alternative, the textured topography can include low aspect ratio lobes or thin planar flakes.

Regardless of the exact process utilized, the operating parameters of the deposition system are selected to tune the morphology and/or composition of the ceramic. In particular, the power applied during the process, total pressure, oxygen/argon ratio and sputter time are controlled. By increasing the power and/or total pressure, the resulting morphology exhibits the high aspect ratio, defined grains that are rougher and crystalline, as described above. In contrast, by decreasing these parameters, the coating becomes more globular and less rough, as also described above.

According to one embodiment, the power applied during the process ranges from about 100 to about 700 watts. Alternatively, the power ranges from about 100 to about 350 watts, from about 150 to about 300 watts, from about 340 to about 700 watts, or alternatively from about 400 to about 600 watts. In one implementation, the total pressure ranges from about 1 to about 30 mTorr. Alternatively, the total pressure can range from about 10 to about 30 mTorr, from about 1 to 10 mTorr, or alternatively from about 2 to about 6 mTorr. In accordance with one embodiment, the oxygen partial pressure ranges from about 10% to about 90%. Alternatively, the oxygen partial pressure can range from about 80% to about 90%, such as for defined grain morphologies, or from about 10% to about 40%, such as for globular morphologies.

In one implementation, the deposition time can control the thickness of the ceramic and the stacking of morphological features. According to one embodiment, the deposition time can range from about 30 seconds to about 10 minutes. Alternatively, the deposition can range from about 1 to about 3 minutes. In one embodiment, the overall thickness of the ceramic ranges from about 50 nm to about 500 nm, or alternatively from about 100 nm to about 300 nm. It is understood that the oxygen content can be increased at higher power, higher total pressure, and high oxygen to oxygen ratios.

Alternatively, the external coating 70 can be made up of various metals such as titanium, platinum, gold, tantalum, or any other similar metal. In a further alternative, the external coating 70 is made up of any conductive material that can produce the rice grain structure described above. In one implementation, the metal for the external coating 70 can be sputtered onto the surface using a process called glancing angle deposition technique (“GLAD”). This process can provide defined peaks and valleys in the surface structure of the coating 70 to achieve a textured surface morphology as described above.

In another alternative, the external coating 70 can be made up of a conductive polymer. One example of such a conductive polymer is poly-ethylenedioxythiophene (“PEDT”), which is available from H. C. Starck, located in West Chester, Ohio. Two other examples include polyaniline- and polypyrrole. In one embodiment, a conductive polymer could be applied by spray coating the polymer onto the surface from a solvent and then subsequently structuring or texturing the polymer using a laser.

In a further alternative, the external coating 70 can be made up of a conductive metal such as platinum, gold, or iridium, or any other such conductive metal that can be used for such an external electrode coating.

The external coating 70 can have a thickness ranging from about 10 nm to about 100 nm. Alternatively, the thickness of the coating 70 can range from about 100 nm to about 3,000 nm. In a further alternative, the thickness can range from about 100 nm to about 1,000 nm.

According to one embodiment, the combination of the structured surface 64 and the conductive coating 66 provides two levels of structure to the overall topology of the electrode 60: a larger or “coarser” base structure (the structured surface 64) and a smaller or “finer” outer structure. That is, the structured surface 64 provides a base topography in the micrometer range (varying in height by as much as 100 μm), while the outer topography of the conductive coating 66 is in the nanometer range (having a thickness of no more than 130 nm).

Further, in accordance with one implementation, both levels of structure contribute to the hydrophobic and cell-repelling qualities of the electrode. That is, the combined topography of the structured surface 64 and the conductive coating 66 results in an overall topography that is hydrophobic, thereby resulting in a hydrophobic electrode. As is understood in the art, the hydrophobic qualities cause the electrode to repel proteins and cells, thereby reducing the incidence of the electrode attaching to any tissue in the human body during use.

According to one embodiment, the hydrophobic qualities of the electrode surface are similar to the hydrophobic qualities of certain self-cleaning plants, because the hydrophobicity in both cases is caused by the overall surface topography. One example of such a plant is the Lotus Flower, which exhibits extreme water-repellency or “superhydrophobicity” because of its topography, which is comprised of a hierarchical composition having both a “rough structure” and a “fine structure”.

In another embodiment, the two levels of structure can also result in increased surface area, which makes it possible to deliver electrical current with lower impedance than would be possible with less surface area. In certain implementations, this means that the electrode 60 can have a shorter length and still provide the appropriate electrical current.

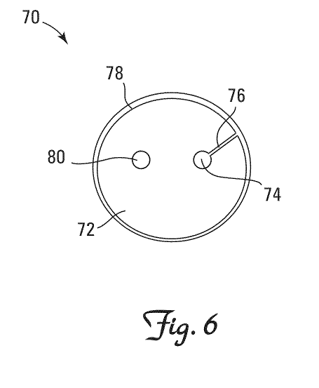

FIG. 6 depicts a cross-sectional view of an electrode 70, according to one embodiment. In this embodiment, the body 72 is a polymeric body 72 that has a lumen 74 in which the conductive wire (not shown) can be positioned. Further, the body 72 can also have a contact channel or lumen 76 in communication with the lumen 74 and the conductive coating 78 of the electrode 70. According to one embodiment, the contact channel 76 provides electrical communication between the lead conductive wire disposed within the polymeric body and the conductive coating 78 of the electrode.

In one embodiment, the polymeric body 72 is a core component of the lead body and is disposed throughout the length of the lead body. Alternatively, the polymeric body 72 is a core component of solely the electrode, such that the electrode having the polymeric body 72 is coupled to the lead body.

In one embodiment, a contact wire (not shown) can be disposed in the contact channel 76 and connected to the conductive coating 78 and the conductive wire to allow for electrical pulses to be transmitted to the electrode 70. In an alternative implementation, application of the adhesion layer for the conductive coating 78 as described above can result in the adhesion layer coating the inner wall of the contact channel 76, thereby providing the electrical communication between the coating 78 and the conductive wire disposed in the lumen 74.

In the embodiment depicted in FIG. 6, the body 72 also has a second lumen 80. As will be appreciated, the lumen 80 can house a second conducting wire or cable, e.g., in a multi-electrode lead. Alternatively, the lumen 80 can be operable as a stylet or guidewire receiving lumen to facilitate lead delivery. As will further be appreciated, the lead body 72 can, in various other embodiments, include three or more lumens depending on the particular functionality desired.

Another embodiment relates to the method of making an electrode having a polymeric or non-polymeric conductive body with a structured surface and a conductive coating similar to the various electrode embodiments disclosed above. In one aspect, the process includes forming a polymeric body having a lumen, structuring the surface of the polymeric body, adding an adhesion layer to the structured surface, adding a conductive external coating over the adhesion layer, and electrically coupling the external coating to a conductive wire disposed in the lumen of the polymeric body. Alternatively, in various implementations in which the lead body is non-polymeric, the process includes forming a metal body having a lumen, structuring the surface of the metal body, and electrically coupling the body to a conductive wire disposed in the lumen of the lead body.

The polymeric body can be formed by any suitable process, whether now known or later developed. In accordance with one implementation, the process of forming a single or multi-lumen polymeric body includes extruding the lead body according to methods known in the art.

In a further embodiment, the structuring of the body surface and the addition or application of the adhesion layer and external coating can be accomplished by any of the methods disclosed above. In addition, the electrical coupling of the conductive coating and conductive wire disposed in the lumen of the body can also be accomplished by any method discussed above.

The scope of the invention is not meant to be limited in application only to leads for implantation in coronary veins. Application of the disclosed embodiments may also be made to right sided bradycardia or tachycardia leads, or epicardial leads. For coronary venous applications, the disclosed embodiment may also be utilized on a non-electrode portion of the lead body.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A medical electrical lead comprising:
a flexible, elongated polymeric lead body defining at least one longitudinal lumen therethrough;
a conducting wire extending through the at least one lumen;
a connector coupled to the lead body for mechanically and electrically coupling the lead to an implantable pulse generator device; and
an electrode including:
an electrode body including a structured surface; and
a conductive coating disposed on the structured surface, the conductive coating electrically coupled to the conducting wire.

2. The lead of claim 1, wherein the structured surface comprises microsized pillars.

3. The lead of claim 1, wherein the structured surface is a laser-structured surface.

4. The lead of claim 1, wherein the structured surface comprises structures on the structured surface having a depth ranging from about 10 μm to about 100 μm.

5. The lead of claim 1, wherein the conductive coating comprises:
an adhesion layer disposed on the structured surface; and
an external coating disposed over the adhesion layer.

6. The lead of claim 5, wherein the external coating comprises an iridium oxide coating.

7. The lead of claim 5, wherein the external coating has a thickness ranging from about 10 nm to about 1,000 nm.

8. The lead of claim 5, wherein the external coating comprises an iridium oxide coating having a rice grain structure.

9. The lead of claim 5, wherein the adhesion layer has a thickness ranging from about 10 nm to about 30 nm.

10. The lead of claim 5, wherein the adhesion layer comprises titanium.

11. The lead of claim 1, wherein the structured surface and conductive coating create a surface hydrophobicity on the electrode.

12. The lead of claim 1, wherein the structured surface comprises a first tier of topography and the conductive coating comprises a second tier of topography, wherein the first and second tiers of topography create a surface hydrophobicity on the electrode.

13. The lead of claim 1, wherein the structured surface and conductive coating result in the electrode having greater surface area than in the absence of the structured surface and conductive coating.

14. The lead of claim 1, wherein the electrode body is a polymeric electrode body or a non-polymeric conductive electrode body.

15. A method of making an electrode for a medical electrical lead of the type having a flexible polymeric lead body and at least one electrical conducting wire therein, the method comprising:
forming a polymeric electrode body;
forming a structured surface on the polymeric electrode body;
applying an adhesion layer to the structured surface; and
applying a conductive external coating to the adhesion layer.

16. The method of claim 15, wherein forming the structured surface is performed using a laser etching process.

17. The method of claim 15, wherein the applying the adhesion layer comprises applying the adhesion layer by chemical vapor deposition.

18. The method of claim 15, wherein the applying the adhesion layer comprises applying the adhesion layer by physical vapor deposition.

19. The method of claim 15, wherein the structured surface, adhesion layer, and external coating create a surface hydrophobicity on the electrode.

20. The method of claim 15, wherein the structured surface comprises a first tier of topography and the adhesion layer and external coating comprise a second tier of topography, wherein the first and second tiers of topography create a surface hydrophobicity on the electrode.

21. An electrode for use on an implantable medical electrical lead having at least one conducting wire therein, the electrode comprising:
an electrode body comprising:
a structured surface; and
a conductive coating disposed on the structured surface, the conductive coating having a surface topography configured to exhibit hydrophobic behavior,
wherein the conductive coating is configured to be electrically coupled to the lead conducting wire.
